(19) United States
(12) Patent Application Publication
(10) Pub. No.: US 2017/0000416 A1
(43) Pub. Date: Jan. 5, 2017

(54) POINT OF WEAR DEVICE

(71) Applicant: i-CalQ, LLC, Salt Lake City, UT (US)

(72) Inventor: Joel R. EhrenKranz, Salt Lake City, UT (US)

(21) Appl. No.: 15/201,149

(22) Filed: Jul. 1, 2016

Related U.S. Application Data
(60) Provisional application No. 62/187,723, filed on Jul. 1, 2015.

(51) Int. Cl. A61B 5/00; A61N 1/32; A61B 5/0205; A61B 3/11

(52) U.S. Cl. CPC A61B 5/681; A61B 5/4839; A61B 3/112; A61N 1/325; A61B 5/02055; A61B 5/7475; A61B 5/7425; A61B 5/486; A61B 5/0022; A61B 5/0024; A61B 5/7282; A61B 5/0531

(57) ABSTRACT

Wearable diagnostic and treatment devices for performing point of care diagnostic tests for detecting, quantifying, analyzing, and treating at least one biological or environmental condition. Such testing devices are designed to provide rapid, quantitative test results in a point-of-care setting or the like where, in the past, only qualitative or semi-quantitative results have typically been available. Likewise, such devices may eliminate or replace expensive, centralized clinical testing equipment and technical personnel. Such testing devices may include automated data reporting and decision support. Further, such devices may include drug delivery or other treatment options.

### CROSS-REFERENCE TO RELATED APPLICATION

This application claims the benefit under 35 U.S.C. §119(e) of U.S. Provisional Application No. 62/187,723, filed Jul. 1, 2015, which is incorporated in its entirety by reference herein.

### BACKGROUND OF THE INVENTION

A number of electro-mechanical devices worn on the wrist, e.g. watches and activity monitors, are available as consumer products. These devices demonstrate consumer acceptance of real-time and continuous biometric and temporal monitoring, but have modest value due to limited precision, accuracy, and functionality.

Wearable biometric monitoring devices, such as heart rate and activity monitors, have become increasingly popular and affordable. Recent advances in sensor, electronics, and power source miniaturization have allowed personal health monitoring devices, also referred to herein as “biometric devices”, to be offered in small sizes. Among the useful types of biometric information such devices may provide are: heart rate, calorie burn, floors climbed and/or descended, location and/or heading, elevation, ambulatory speed and/or distance traveled, etc.

PDA and smartphone based diabetes monitoring devices have been studied. Generally limited to the context of insulin delivery, such devices allow a user to interface with the device upon collecting blood from a skin prick, analyzing the sample, and utilizing onboard or remote software and communication to facilitate insulin infusion decisions on the basis of diet, physical activity, and the like. Such devices continue to suffer from limited applicability and reliance on user inputs.

There is a need for a new class of biometric devices, that enable acquisition of a diverse variety of biological and environmental information in real time, store, analyze, and process such information for correlation with user and situation specific factors, such as diet and other activities, including physical and/or mental activity, stress and other factors. Furthermore, treatment such as drug delivery in response to appropriate data and analysis should be enabled. Such devices may provide situation and health management by the wearer or in conjunction with other parties, such as health professionals, military superiors, or local officials as necessary.

So-called “smart” devices, such as smart watches developing out of smartphone and computer technology, have recently also become available. Such devices may combine the power of modern mobile computing with the high level of portability and fashion conscious elements of wearable devices. With WiFi, Bluetooth, and other connectivity platforms available, smart watches boast software application capability, data storage, communications, displays, user interfaces, camera, gyroscopes, and activity monitors, etc. As such, smart watches and other similar wearable devices are well suited as development platforms to meet the above described need.

### BRIEF SUMMARY

The following disclosure describes a number of embodiments that use wrist electro-mechanical devices for monitoring and treatment of medical conditions. The monitoring and delivery systems thereof are intended to cover a broad range of health problems and environmental detection, along with a variety of potential treatments, and is not to be construed as being limited to the specific applications disclosed herein.

Wearable diagnostic and treatment devices for performing point of care diagnostic tests for detecting, quantifying, analyzing, and treating at least one biological or environmental condition are disclosed herein. Such wearable testing devices are designed to provide rapid, quantitative test results in a point-of-care setting or the like where, in the past, only qualitative or semi-quantitative results have typically been available. Likewise, such devices may eliminate or replace expensive, centralized clinical testing equipment and technical personnel. Such devices may include automated data reporting and decision support. Further, such devices may include drug delivery or other treatment options.

In an embodiment, a wearable device for monitoring and treating medical conditions is disclosed. The device includes a housing and a means for securing the device to the user, such as a watch band or other strap. The device housing may contain one or more sensor components, a user interface, software, communication capability, and treatment options which may also be disposed in the securing device. The wearable device may also include a testing device that has data collection and data analysis capabilities. In an embodiment, the wearable device may be an electromechanical device, such as a smartwatch.

In another embodiment, the wearable device sensor component is configured to detect and monitor one or more biometric or environmental signals including, but not limited to pupillary constriction, skin conductivity, blood pressure, blood oxygenation level, respiration rate, radial artery temperature, heart rate, heart rhythm, interstitial fluid, capillary blood, dangerous gas detection.

In further embodiments, the user interface of the wearable device may be a graphical user interface (GUI). Such a GUI may be configured to accept user input and display data collected by the sensor component, data received via communication with database servers and/or qualified experts, or display treatment recommendations, and instructions.

In another embodiment, the medical condition being monitored or tested may be one or more of a group of conditions that may be tested by a wearable device of the disclosure including, but not limited to, nerve gas exposure, anaphylaxis, opiate overdose, hypoglycemia, hypertension, hypotension, heart arrhythmia, asthma, bacterial sepsis, pulmonary embolism, and air contamination by poisonous gasses.

In still another embodiment, the communications component of the wearable device comprises at least one of many known technologies available to smartphones and laptop-type portable devices, such as wired internet connection, WiFi internet capability, Bluetooth capability, and cellular capability.

In another embodiment, the treatment component of the wearable device may include, alone or in combination, at least one of drug delivery, treatment recommendation, and treatment instructions communicated to the user via the communications component and/or display on the user interface.

### BRIEF DESCRIPTION OF THE FIGURES

FIG. 1 illustrates a top view of a wearable diagnostic device according to one embodiment of the present disclosure.

FIG. 2 illustrates a side view of a wearable diagnostic device according to one embodiment of the present disclosure.

### DETAILED DESCRIPTION

Wearable diagnostic and treatment devices for performing point of care diagnostic tests for detecting, quantifying, analyzing, and treating at least one biological or environmental condition are disclosed herein. Such devices are designed to provide rapid, quantitative test results in a point-of-care setting or the like where, in the past, only qualitative or semi-quantitative results have typically been available. Likewise, such devices may eliminate or replace expensive, centralized clinical testing equipment and technical personnel. Such testing devices may include automated data reporting and decision support. Further, such devices may include drug delivery or other treatment options.

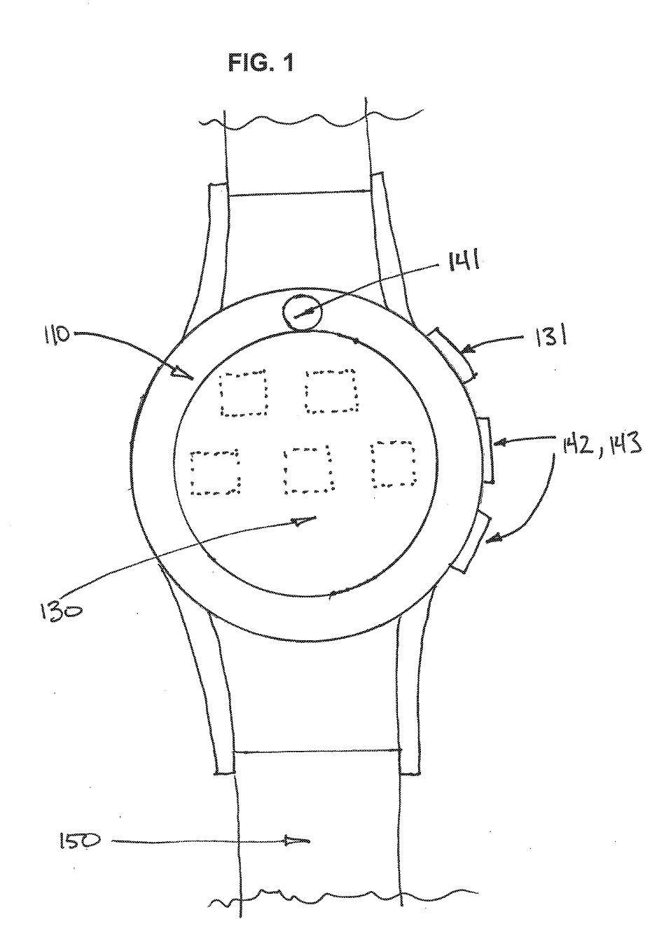

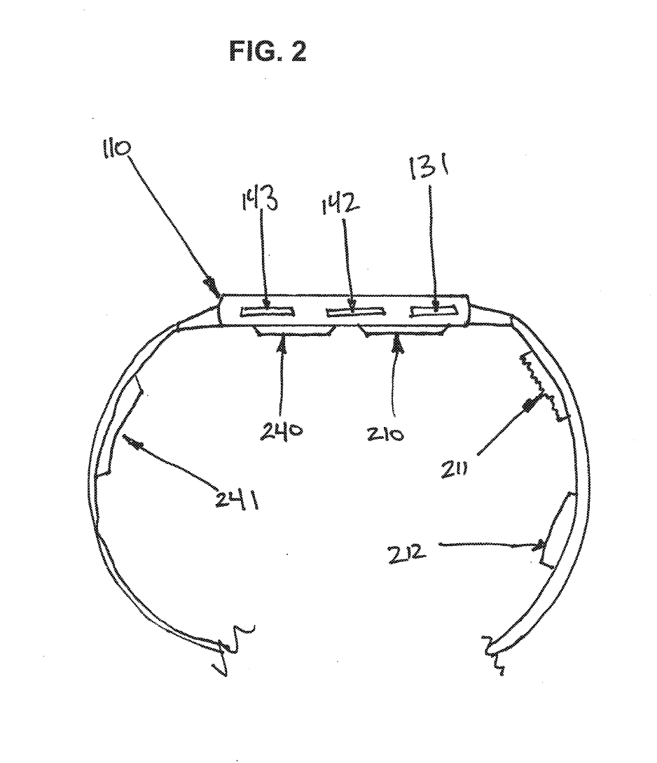

In an embodiment as shown in FIGS. 1 and 2, a wearable device 100 for monitoring and treating a medical condition is disclosed. The device includes a housing 110 and a means for securing 150 the device to the user, such as a watch band or other strap. The device housing 110 may contain one or more sensor components 141, 142, 143, 240, 241, a user interface 130, 131, software, communication capability, and treatment options 210-212, which may also be disposed in the securing device 150, for example, a watch band. The wearable device may also include a testing device that has data collection and data analysis capabilities. In an embodiment, the wearable device may be an electromechanical device, such as a smartwatch.

Sensor components of the wearable device may include those known in the art, adapted for use in portable, miniaturized devices. Such sensors may be imaging detectors 141 capable of collecting and storing electromagnetic radiation, including but not limited to digital cameras and infrared detectors. See, for example, Kim, et. al., J. Opt. Soc. Korea 2013, 17(3), 249, the contents of which are incorporated by reference.

Certain sensors 241 may be incorporated into the wearable device through use of a securing component such as a wrist strap 150, or alternatively may be in communication with the wearable device, such as with a chest strap that is in communication with a communication component. Further securing means may include ankle, finger, and waist bands. The device should be secured to minimize movement artifacts and to position device sensors and effectors in proximity to biometric signal sources. Such securing means may also include integration of the device with clothing, e.g. heart and ECG monitor bra straps, or shirts with blood pressure cuff sleeves. Art recognized sensors of this type may include those that detect and measure blood pressure, heart rate, and heart rhythm, and respiration rate. See, for example, US Patent App. Publication 20140142403, the contents of which are incorporated by reference.

Blood oxygenation levels are an important indicator of a variety of medical conditions and can be included in the wearable device. Body and core temperature may also be detected via wrist sensors 240, such as sensors positioned on the wrist proximate to the radial artery. See, for example, US Patent App. Publication 20070197887, the contents of which are incorporated by reference. Sensors capable of quantifying oxygenation in blood through non-invasive methods are well known in the art and may be incorporated into embodiments of the disclosure. See, for example, US Patent App, Publication 20080208009, the contents of which are incorporated by reference. Dangerous gasses in the atmosphere, such as mine gas, or unhealthy levels of carbon dioxide and other gasses may be detected via art recognized sensors 142 incorporated into embodiments of the disclosure. See, for example, U.S. Pat. No. 7,528,711, the contents of which are incorporated by reference. Further, various sensors may be included in the device which may detect and analyze tests and samples collected or prepared external to the device itself. Art recognized samples and tests of this nature include, but are not limited to, lateral flow assays, blood glucose tests, interstitial fluid tests, and the like. See, for example, US Patent App. Publication 20120190955, the contents of which are incorporated by reference.

In order to process, interpret, present, and organize raw data detected and generated by the sensors of the present disclosure, it will be recognized that smart watches, like smart phones and laptops, will necessarily incorporate software applications capable of accomplishing the required tasks. Such software may include applications capable of image processing, database manipulation, remote server communication, driving the incorporated sensors, comparing observed data to known standards, assessing risk factors, comparing data streams from more than one sensor for correlations, and the like.

As with art recognized smart devices, the wearable smart device of the present disclosure comprises communications capability. Such communications components of embodiments disclosed herein may include wired and WiFi internet connection, cellular connection, Bluetooth capability, and the like. Communications with and between the disclosed device and its user, and database servers and/or qualified providers and experts are thus enabled.

It will be appreciated that appropriate medical treatment decisions necessarily involve detection and analysis of various biometrics, potentially combined with knowledge of practitioners and specialists, along with the patient/user themselves. The wearable device of the present disclosure may be configured to facilitate and implement treatment by the device alone, or through some combination of internal software analysis, communication with providers and/or databases, and user interface inputs. Such treatments may include any art recognized automatic drug delivery from, for example, containers 210, 211, 212 or blister packs incorporated into the user securing means 150, such as a watch strap. See, for example, U.S. Pat. No. 6,745,071, the contents of which are incorporated by reference. Such drug delivery is well known in the context of insulin delivery to diabetic patients, and may be applied to readily dispensed drugs to treat conditions of the disclosed embodiments. Alternatively, drug delivery may be performed via a communications component that sends dosing instructions to a remote device (e.g., an insulin pump or a medication pump). Drug delivery may be accomplished by way of epidermal delivery, transdermal delivery, intravenous delivery, or any other know suitable delivery method that provides drug delivery via a wearable device, such as, for example, through a transdermal matrix or a needle. Further treatment may include instructions delivered via communications with providers or databases via the user interface of the disclosure. Treatment may be automated in some embodiments, or triggered by user or provider decision making, whether remote to the user or on-site.

Embodiments of the wearable device may include a user interface 130, 131 configured to control the functions of the device and display data and communications to the user. Functions common to wearable devices, such as smart watches, may be incorporated into the disclosure. Such functions include, but are not limited to, a graphical user interface. Such interfaces may be touch sensitive in order to change display, input user data, operate controls of incorporated sensors and software, operate testing components, operate the incorporated camera, operate communications components, initiate treatment, and the like.

Further, it is understood that such wearable smart devices may be configured such that each of the incorporated components within the device housing, securing means, and external testing elements are operably coupled to one another through electro-mechanical means. That is, the sensors, software, communications, treatment, and user interface components can all operate together as a combined device to diagnose, analyze, and treat medical conditions that may be assessed by the wearable device of the disclosure.

Non limiting examples wherein combinations of the various disclosed embodiments may be employed to diagnose, monitor, and treat health conditions are as follows:

Nerve gas detection and treatment: The mechanism of action of nerve gas is inhibition of acetylcholinesterase. This results in increased cholinergic activity, of which one of the earliest signs is constriction of the pupils. Soldiers in combat zones at risk for nerve gas exposure have a pre-filled syringe with atropine on their persons at all times. The current procedure for detecting nerve gas exposure consists of recognizing a pattern of arm waving and hearing warning shouts. The response is to don a protective suit and respirator and inject atropine if exposure occurs. An alternative method for detecting nerve gas of the present disclosure may be using the front facing camera on a watch to monitor pupillary size. Miosis (pupillary constriction) is one of the earliest manifestations of nerve gas exposure. The watch camera can detect miosis as a sign of nerve gas exposure. Another sign of nerve gas exposure is increased sweating. This could be monitored by measuring skin conductivity using electrodes embedded in the watch band or watch back. When signs of nerve gas exposure are detected, the device can auto-inject atropine from, for example, a blister pack contained in the watch band. In the case of accidental/excessive atropine injection, another blister pack containing physostigmine or pilocarpine (atropine antagonists) could be administered.

Anaphylaxis detection and treatment: Anaphylaxis is characterized by hypotension, and histamine and IgE release into the circulation. Wrist blood pressure monitors are well known. Watch wrist bands could be modified to measure blood pressure. Histamine and IgE can be measured in a variety of body fluids, including saliva, interstitial fluid, and blood, using a lateral flow immunoassay. The wrist device can obtain interstitial fluid by iontophoresis, perform a lateral flow assay for histamine or IgE, or the individual could collect a blood or saliva sample and perform the test themselves. Another sign of anaphylaxis is bronchoconstriction; this results in decreased blood oxygen saturation. The wrist band can include, for example, an infrared oxygen saturation sensor. Upon detecting signs of anaphylaxis (hypotension, decreased oxygen saturation, and/or increased histamine and/or IgE), the device can deliver epinephrine, an antihistamine, and/or a glucocorticoid from a prefilled container on the band.

Opiate overdose: Opiate overdose is characterized by miosis and hypoventilation. This can be iatrogenic, due to medical error, or due to exposure to aerosolized opiates (e.g. fentanyl, as was used in the Moscow hostage rescue attempt:

https://en.wikipedia.org/wiki/Moscow_hostage_crisis_chemical_agent). Using the front facing camera on a watch, miosis can be detected. Monitoring oxygen saturation (as described above) and respiratory rate monitoring (through any number of known optical and acoustic techniques, such as US Patent App. Publication 20140081100, which is incorporated herein by reference) can identify opiate overdosage. A container including an opiate antagonist such as naloxone on the securing strap or watchband can be administered to reverse the opiate overdose.

Hypoglycemia can be detected by sensing a fall in core temperature in the radial artery (e.g., with a watch sensor positioned directly over the radial artery) and detecting interstitial glucose levels. In such cases, the securing means can include a standard treatment for hypoglycemia, such as glucagon.

Hypertension can be monitored as described above. Significant blood pressure elevations could be treated with phentolamine or any other rapid acting hypotensive agent, that is disposed within a container on the wearable device (e.g., on the wristband). Additionally, blood pressure trends, along with indices such as blood oxygen saturation can be monitored over time. Sleep apnea, pheochromocytoma, and metabolic syndrome can thus be diagnosed. Upon such diagnosis, a transdermal anti-hypertensive such as clonidine or a combination of drugs may be administered from the wearable device.

Hypotension could be monitored as well. In patients with orthostatic hypotension due to diabetes or Parkinson's Disease, postural falls in blood pressure are a major clinical problem. The watch can detect a drop in blood pressure associated with a change in posture (detected by watch accelerometer or gyroscope) and administer a rapid acting pressor (e.g. dopamine, or levophed) from a container on the watch band. In patients with adrenal insufficiency or Addison's disease, the hypotension can be treated with dexamethasone.

By monitoring heart rate and rhythm using electrodes on the device, arrhythmias can be detected. These can be treated by the administration of a rapid acting anti-arrhythmic agent, such as amiodarone, adenosine, or lidocaine. This would replace an implantable automatic defibrillator.

Asthma: Status asthmaticus is a life threatening emergency. This can be detected by increased heart rate, respiratory rate, and decreased oxygen saturation. Treatment by the wearable device of the disclosure may consist of administration of epinephrine, dexamethasone, and/or other anti-bronchospastic agents.

Bacterial sepsis can be monitored by serial measurement of procalcitonin (from interstitial fluid or capillary blood) and infrared core temperature monitoring from the radial artery. Other art-recognized markers of sepsis such as c-reactive protein (CRP), white blood cell count, soluble membrane attack complex, and interleukin(s) can be similarly monitored. Treatment by the wearable device of the disclosure may consist of administration of a broad spectrum antibiotic, such as ceftriaxone.

Pulmonary embolism could be detected by monitoring oxygen saturation, heart and respiratory rate, and d-dimer concentration. Treatment may consist of dispensing heparin from a prefilled container disposed on the device.

Detection of mine gas or an air contaminant in a mine: Hazardous gasses that accumulate in mines when subsurface strata are exposed include, but are not limited to, methane, carbon dioxide and related gases, hydrogen sulfide, and carbon monoxide. Dangerous gases can be detected by including sensors for the various gases in the wearable device, such as on the watch face, watch band, or watch back. Detection of dangerous gases can be linked to activation of ventilation systems, warning sirens, and the like.

The present disclosure may be embodied in other specific forms without departing from its spirit or essential characteristics. The described embodiments are to be considered in all respects only as illustrative and not restrictive. The scope of the disclosure is, therefore, indicated by the appended claims rather than by the foregoing description. All changes which come within the meaning and range of equivalency of the claims are to be embraced within their scope.

1. A wearable device for monitoring and treatment of a medical condition, comprising:
a device housing and means for securing the housing to a user;
a sensor component;
a user interface disposed on a primary face of the device housing;
a software component operatively coupled to the sensor component and configured to display data upon the user interface;
a communication component operatively coupled to the software component; and
a treatment component disposed within the device housing or securing means and operatively coupled to the software component, configured to deliver medication to the user.

2. The wearable device of claim 1, wherein the device is a digital-electronic wrist watch.

3. The wearable electro-mechanical device of claim 2, wherein the wristwatch is a smartwatch.

4. The wearable device of claim 1, wherein the sensor component is configured to detect and monitor at least one of pupillary constriction, skin conductivity, blood pressure, blood oxygenation level, respiration rate, radial artery temperature, heart rate, heart rhythm, interstitial fluid, capillary blood, and atmospheric gas.

5. The wearable device of claim 1, wherein the user interface is a graphical user interface (GUI).

6. The user interface of claim 5, wherein the user interface is configured to accept user input.

7. The user interface of claim 5, wherein the user interface is configured to display data collected by the sensor component, communicated data, treatment recommendation, administered medication, and instructions.

8. The wearable device of claim 1, wherein the medical condition is at least one of nerve gas exposure, anaphylaxis, opiate overdose, hypoglycemia, hypertension, hypotension, heart arrhythmia, asthma, bacterial sepsis, pulmonary embolism, and air contamination.

9. The wearable device of claim 1, wherein the communications component comprises at least one of wired internet connection, WiFi internet capability, Bluetooth capability, cellular capability.

10. The wearable device of claim 1, wherein the treatment component comprises at least one of drug delivery, treatment recommendation, and treatment instruction.

11. A method of detecting, monitoring, and/or treating a medical condition at the point of care, the method comprising:
providing a wearable device configured to detect, monitor, and/or treat the medical condition;
monitoring at least one biological or environmental, signal;
assessing data obtained by monitoring the at least one biological or environmental signal;
determining treatment for the medical condition; and
treating the medical condition.

12. The method according to claim 11, wherein monitoring the at least one biological signal comprises detecting at least one of pupillary constriction, skin conductivity, blood, pressure, blood, oxygenation level, respiration rate, body temperature, heart rate, heart rhythm, biomarkers in interstitial fluid, capillary blood, exhaled air, and atmospheric gas.

13. The method according to claim 11, wherein assessing date comprises comparing the data to known standards and/or previously collected user data.

14. The method according to claim 11, wherein determining treatment for the medical condition comprises at least one of software analysis of the assessed data, communication with remote databases or skilled providers, and user self-determination.

15. The method according to claim 11, wherein treating the medical condition comprises at least one of drug delivery to the user from a container disposed on the wearable device or directly or electronically linked to the wearable device, displaying a treatment recommendation, and alerting a skilled provider.

16. The method according to claim 11, wherein the medical condition is hypertension.

17. The method according to claim 16, wherein monitoring the biological condition comprises measuring blood pressure.

18. The method according to claim 16 wherein treating the medical condition comprises administering an anti-hypertensive drug disposed within a container on the wearable device or connected directly or electronically to the wearable device.

19. The method according to claim 11, wherein the medical condition is opiate overdose.

20. The method according to claim 19, wherein monitoring the biological condition comprises at least one of detecting miosis, monitoring oxygen saturation, and monitoring respiratory rate.

21. The method according to claim 19 wherein treating the medical condition comprises administering an opiate antagonist drug disposed within a container on the wearable device.

22. The method according to claim 11, wherein the wearable device is the device according to claim 1.
